(19) United States
(12) Patent Application Publication
(10) Pub. No.: US 2007/0162153 A1
(43) Pub. Date: Jul. 12, 2007

(54) Prosthetic sock providing graduated thickness and tibial crest load reduction for an amputee limb remnant

(76) Inventors: Leslie James Barnes, Hurstbridge (AU); Richard John Law, Croydon (AU)

Correspondence Address: DOROTHY S. MORSE, 515 PARK DRIVE, NW, BRADENTON, FL, 34209, US

(21) Appl. No.: 11/326,733

(22) Filed: Jan. 7, 2006

(51) Int. Cl. A61F 2/78

(52) U.S. Cl. 623/36

(57) ABSTRACT

A prosthetic sock configured to accommodate a transtibial amputee's residuum volume reductions which occur over time and are greater distally than proximally. It is also applicable to other amputee limbs and other levels of amputation of the leg and arm. The sock provides better compensation for the increased distal volume shrinkage and it reduces discomfort from localized loading on the stump by providing a knitted thickness that is greater distally and then tapers to a lesser proximal knitted thickness. A transtibial amputee's residuum is more sensitive to pressure over the tibial bone and this sock also provides a reduced local thickness over the tibial crest area which decreases the localized pressure in this region. Since the sock accommodates the loss of tissue volume in the distal end of the prosthetic socket, it thereby provides improved stump stability inside the prosthetic socket and improves amputee control and stability in walking.

### BACKGROUND OF THE INVENTION

1. Field of the Invention

This invention relates generally to the field of prosthetic devices, and more specifically to a prosthetic sock having an integral thicker knitted portion wherein the greatest thickness in the sock is positioned distally near its closed end, with a tapering graded reduction of thickness moving proximally toward its open end, providing increased loading distal to the amputee limb remnant soft tissues while reducing the loading over the amputee's tibial crest area which is more sensitive to pressure. The sock accommodates a transtibial amputee's residuum volume reductions which are greater distally than proximally, although it is also applicable to other amputee limbs and other levels of amputation of the leg and arm. Since the sock provides better compensation for the increased distal volume shrinkage and a reduced local thickness over the tibial crest area, it reduces discomfort from localized loading on the transtibial amputee's residuum. Further, since the sock makes accommodates the loss of tissue volume in the distal end of a prosthetic socket, it thereby provides improved stump stability inside the prosthetic socket and improves amputee control and stability in walking.

2. Description of the Related Art

A transtibial amputated limb residuum, or stump, normally experiences volume reductions, which can have a dramatic effect upon the fit of an artificial prosthesis. It was stated by Marks, L. J. and Michael, J. W. (2001) Clinical review; Science, medicine, and the future—Artificial limbs. British Medical Journal; 323:732-735 that “The single most critical aspect of any prosthesis is the quality of the interface between the limb remnant (stump) and the artificial prosthesis.” However, in discussing the Transtibial amputee's stump volume changes it was also said, “being a dynamic organ, the stump tends to shrink (atrophy) over time . . . ”

All amputees have limb tissue with substantial volume reductions. In Beers and Berkow (eds.) (2000) The Merck Manual of Geriatrics, Merck & Co., Inc. Ch 29 it was stated that “The stump usually continues to shrink with use. An adequate fit of the prosthesis can be achieved by adding layers of socks, although eventually a new socket is needed.”

Current stump/prosthetic socks are of consistent and equal knitted thickness throughout their length and circumference. While they may be manufactured with a conical shape, or even more complex shape (eg bulbous), no attempt has been made to address the issue of uneven shrinkage throughout the length of the residuum.

For recently amputated people, as the residual limb becomes looser in the prosthetic socket, it is a practice for the prosthetist and other allied health professionals to recommend the addition of one or more stump socks to compensate for the reduced stump volume. The selection and application of stump socks is taught to the amputee as part of their rehabilitation to allow them the independence to monitor their limb remnant and manage themselves. However, selection of the appropriate number and thickness of stump socks can be difficult and many amputees have injured their residuum by creating situations where the prosthesis is too tight or too loose, simply as a result of inappropriate stump sock selection. Further, the shrinkage difference between the distal end volume and the proximal volume in the knee joint region may be so large, that the use of even thickness stump socks cannot accommodate the difference. The amputee must then persevere with a prosthesis that is too loose distally and comfortable proximally, or the reverse situation where a prosthesis is too tight proximally and comfortable distally. Sometimes the compromise to maintain socket stability is for amputee to experience discomfort in both the proximal and distal regions of the residuum.

The current design of stump/prosthetic socks to compensate for volume fluctuations in the residuum, assumes that the shrinkage and atrophy of the stump occurs equally within the prosthetic socket. This is currently the most common form of volume reduction management. However, the distal end of the transtibial stump typically has much greater stump shrinkage, from both oedema reduction and soft tissue atrophy, than the proximal end. Greater distal end stump shrinkage occurs initially following amputation as oedema subsides and continues to a lesser degree throughout the transtibial amputee's life because of muscle atrophy. Normal stump socks do not manage this volume reduction adequately, as they apply a uniform sock thickness to the whole limb remnant and do not allow for the increased distal reduction.

Amputees who use a prosthetic socket with a polyethylene foam (Pelite) liner, may have the liner packed with thin patches of pelite material to take up any perceived slackness inside the socket. The disadvantage of this system is that it requires a careful estimation by the amputee of the appropriate amount of packing and is prone to over or under packing error. Compensation for distal end volume reduction can also be achieved by using a “half sock” which is a cut sock applied to the distal end of the stump. However, this creates a ridge line at the level of the residuum where the sock is cut, and can easily cause tissue damage at the cut-line site because of differential tissue pressure each side of the cut line.

Another different and more complex approach to the increased distal reduction in a residuum uses a liquid filled bladder built into the prosthetic socket. However, this approach requires highly sophisticated socket design processes and complex construction methods. Other complex systems of inflatable bladders and adjustable socket systems have also been developed. However, they have the disadvantage of being prone to puncture and make the prosthesis heavier. Thus, there was a need to provide a stump/prosthetic sock that more adequately accommodated the uneven transtibial limb remnant shrinkage.

U.S. Pat. No. 4,635,626 to Lerman (1987) discloses a sock that attempts to cushion loads on an amputee's stump and reduce shear forces by providing a gel material between knitted cloth layers. However, this design is complex and expensive to manufacture. Further, no attempt to allow for tissue volume reduction is included in its design. In contrast, the present invention provides accommodation for such tissue volume reduction, while reducing the loading over the amputee's tibial crest area which is more sensitive to pressure. There is no prosthetic device known with the same structure of the present invention, or all of its advantages.

### BRIEF SUMMARY OF THE INVENTION

It is therefore the primary objective of the present invention to provide a stump sock design with greater distal thickness tapering to a thinner proximal thickness, and which is configured to accommodate the greater distal limb remnant volume reduction that naturally occurs over time in an amputee's residuum. It is also the objective of the present invention stump/prosthetic sock to provide a soft, compliant and flexible interface that assumes the shape of an amputee's residual limb so that sensitive skin tissues are not exposed to abrasion or local pressure points. It is a further objective of the present invention stump/prosthetic sock to provide an inner sock surface with moisture transport capability that allows wicking of moisture away from the skin. It is also the objective of the present invention stump/prosthetic sock to provide an outer sock surface with a smooth and durable surface that absorbs and holds moisture away from the limb remnant. In addition, it is also an objective of the present invention stump/prosthetic sock to provide a sock that accommodates the greater relative prominence of the tibial crest, so that reduced sock thickness over that localised area is achieved. In a still further embodiment of the present invention there is provided for an ankle amputation having a stump sock with a reduced thickness in the knitted material positioned over the bony crest of tibia to reduce discomfort from localised loading over that bony crest.

When properly made and used, the present invention provides a stump/prosthetic sock capable of better compensation than known prosthetic socks for amputee limb remnant distal volume reduction, particularly for transtibial amputee stumps, by providing a sock with a greatest knitted thickness distally, which then tapers to a lesser proximal knitted thickness. Thus, in accordance with the present invention, there is provided a prosthetic sock comprising: a tapered thickness sock for an amputee limb remnant that has a soft and preferably knitted integral thickness that is greater in the portion of the sock positioned distally near its closed end and has a tapering graded reduction of thickness moving proximally toward its open end, which provides increased loading distal to the amputee limb remnant soft tissues while reducing the loading over the amputee's tibial crest area which is more sensitive to pressure. This invention provides compensation for non-uniform stump shrinkage, in which greater shrinkage normally occurs at a residual limb's distal end. The compensation achieved by the present invention can result in a more correct interface between the limb remnant and a prosthesis socket, giving improved comfort for the amputee, improved amputee control and stability in walking, and a longer useful life for the stump sock, socket liner, and associated prosthesis when compared with previous stump sock designs. Advantageously, the present invention provides a range of stump sock sizes and thicknesses having differing wall thicknesses at or adjacent their distal ends such that as stump shrinkage proceeds, a progressively greater degree of compression can be provided by changing to different thickness socks. This is a superior method of stump management than current arrangements where multiple uniform thickness socks are used and the wall thickness at the proximal end generally remains constant between socks of differing distal wall thickness.

### BRIEF DESCRIPTION OF THE SEVERAL VIEWS OF THE DRAWINGS

Exemplary embodiments of the invention will now be described in accordance with the accompanying drawings, as follows.

FIG. 1 is a front view of the layout of the positioning of bands in the most preferred embodiment of the present invention sock.

FIG. 2 is a front view of the thickness gradient provided in the bands of the most preferred embodiment of the present invention sock.

FIG. 3 is a cross-sectional view of the socks in the most preferred embodiment of the present invention sock.

FIG. 4 is a table providing details of the lengths and thicknesses of the regions for preferred small, medium and large embodiments of the present invention sock.

### DETAILED DESCRIPTION OF THE INVENTION

The most preferred embodiment of the present stump/prosthetic sock is open on its proximal end 1 to allow the introduction of an amputee's limb remnant (not shown) and has a thicker knitted closed distal end 6 with a knitted loop construction having elasticised fibres allowing limb remnant shape accommodation without wrinkles and a soft cushion inner surface. Between proximal end 1 and distal end 6, the most preferred embodiment of the present stump/prosthetic sock invention has a tapering graded thickness reduction via bands 2-5 that provides increased loading to the amputee limb remnant soft tissues while reducing the loading over the amputee's tibial crest area which is more sensitive to pressure.

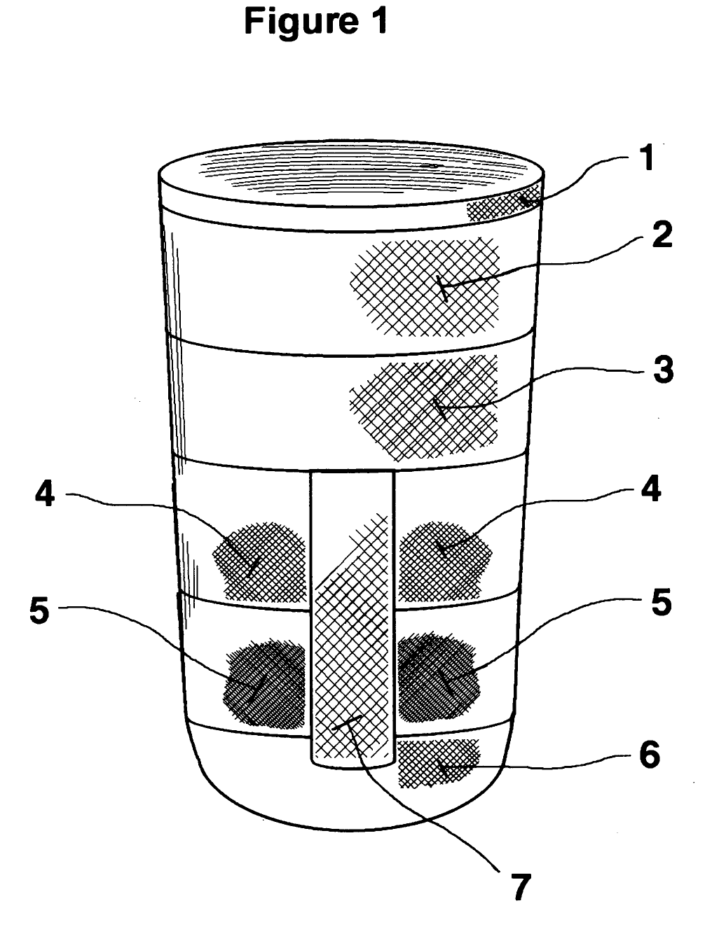

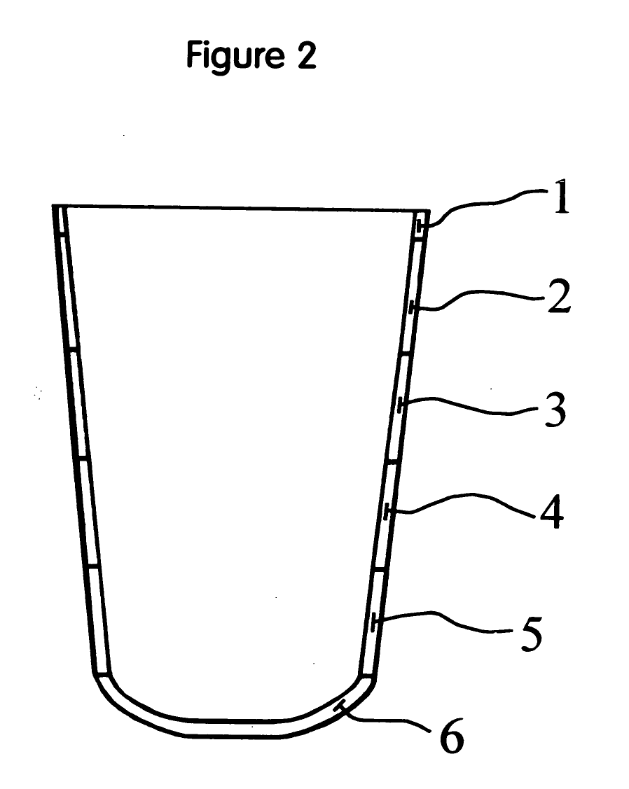

FIGS. 1 and 2 show the present invention sock having six circumferential bands of differing length and thickness dimension, identified by the numbers 1, 2, 3, 4, 5, and 6. While the length dimension of bands 1-6 shown is preferred, variations may occur in some applications, such as for the residual limb of a child. The circumferential bands graduate in thickness from a greatest thickness dimension in its closed distal end 6 to a lesser thickness dimension in its proximal end 1. FIG. 1 also shows a vertical region 7 that is an approximately 30 mm wide strip on the front or anterior portion of the present invention sock. The anterior portion is substantially thinner than bands 1-6, which is contemplated for positioning over the particularly sensitive tibial crest area of the amputee's limb remnant. Bands 1-6 and anterior portion 7 preferably have a knitted construction, and in the most preferred embodiment of the present invention prosthetic sock, band 6 has a knitted loop construction of cotton, wool, polyester, and elastomeric fibres.

Although not strictly limited thereto, with other embodiments of the present invention having some variation thereto, the construction of the most preferred embodiment of the present invention has the following configurations and dimension. It is contemplated for the distal band 6 of the most preferred embodiment to have an approximate 3.4 mm thickness dimension and be made from a medium weight or density terry loop pile material. Distal band 6 is also designed to conform closely to the shape of the amputated limb remnant end. Band 5 in the most preferred embodiment of the present invention has an approximate 4.8 mm thickness dimension and is made from a thick knitted terry loop pile material. It is contemplated for band 5 to have the greatest wall thickness, to correspond to the distal region of the limb remnant which experiences the greatest volume reduction. It is further contemplated for band 4 in the most preferred embodiment of the present invention to have an approximate 3.4 mm thickness dimension and be made from a medium weight or density knitted terry loop pile material. Since a little less limb tissue reduction occurs in this region of the residuum, less sock thickness is needed in this region. Band 3 in the most preferred embodiment of the present invention would correspondingly have an approximate 2.4 mm thickness dimension and be made from a thin knitted terry loop pile material. Even less limb tissue reduction occurs in this area of the amputated limb and less sock thickness is needed in this region. Band 2 in the most preferred embodiment of the present invention would correspondingly have an approximate 1.8 mm thickness and be made from material with a plain knit structure. This region corresponds to the most proximal limb remnant region near the anatomical knee joint where minimal tissue volume reduction occurs. Further, since Band 1 in the most preferred embodiment of the present invention is never inside the transtibial prosthetic socket, it is contemplated for band 1 to comprise a double plain welt structured to provide strong reinforcement and enable better hand gripping for sock donning onto the limb remnant. Elastic fibres may also be incorporated into band 1 to assist in retaining its position on the stump. It is the graduated wall thickness from the thickest band 5 through bands 4 and 3, and then through band 2, that is designed to provide for the non-uniform shrinkage that occurs in an amputee's limb remnant.

The vertically-extending band 7, also referred to herein as the anterior portion or tibial channel, has an approximate 1.8 mm thick dimension and is made from a material having a plain knitted structure that is approximately 30 mm in width. Band 7 is designed to be positioned above the crest of the tibia and be generally limited to that region. The tibial channel of band 7 in the most preferred embodiment of the present invention extends from approximately one centimetre into band 6, through the entirety of band 5 and substantially through band 4. Further, it is contemplated for the length of tibial channel of band 7 to vary from approximately 110 mm in length for a small prosthetic sock to approximately 140 mm in length for a large prosthetic sock. The areas of bands 4 and 5 in contact with the tibial channel of band 7 have a greater thickness dimension than band 7, with the maximum difference in thickness being approximately 3 mm. Band 7 in the most preferred embodiment of the present invention is also designed to extend from the bone end of the limb remnant, to the tibial tuberosity proximally. The most preferred embodiment of the present invention sock can be manufactured by knitting it on a single cylinder sock knitting machine ranging between approximately 90 and 125 mm in diameter and having approximately 96 to 136 needles. Between each needle, the machine has pairs of sinkers, one for knitting and the other, having a higher nib, for forming the internal terry loops of the sock. To include the tibial channel of band 7, the latter must be capable of being selected into at least two groups to enable knitting of the tibial channel/band 7 region.

To be effective as a stump/prosthetic sock, the knitting yarn produces a present invention sock that is soft, moisture absorbent and resilient on the inside. The terry loops on the inside of the present invention sock provide a high cushioning effect and the yarn provides a wicking of moisture away from the skin to the outside of the sock. Further, the outside of the sock is smooth, durable and capable of absorbing and holding moisture. The sock of the present invention also has sufficient elasticity to conform to an amputee's limb remnant and provide a smooth, wrinkle free fit onto the amputee's limb.

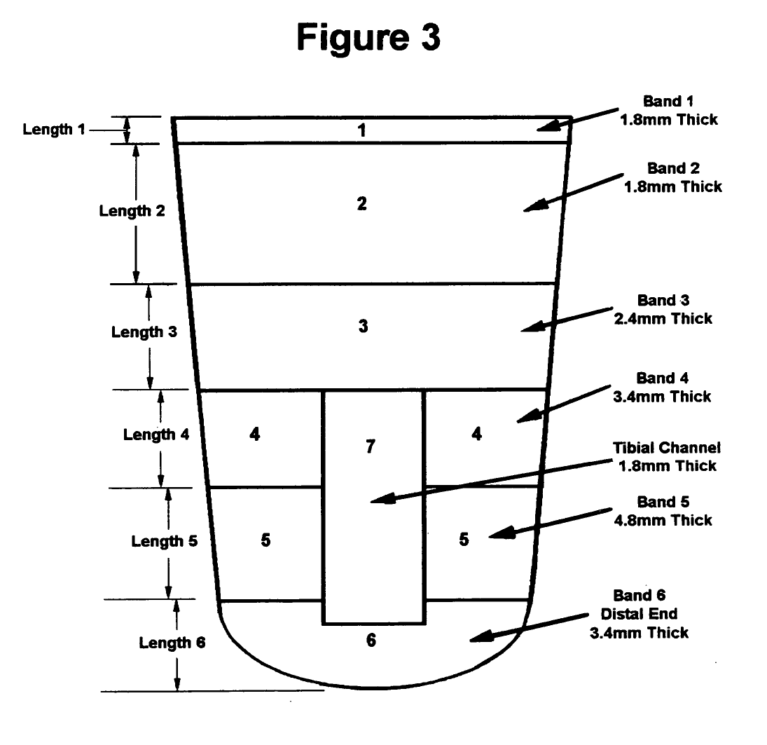

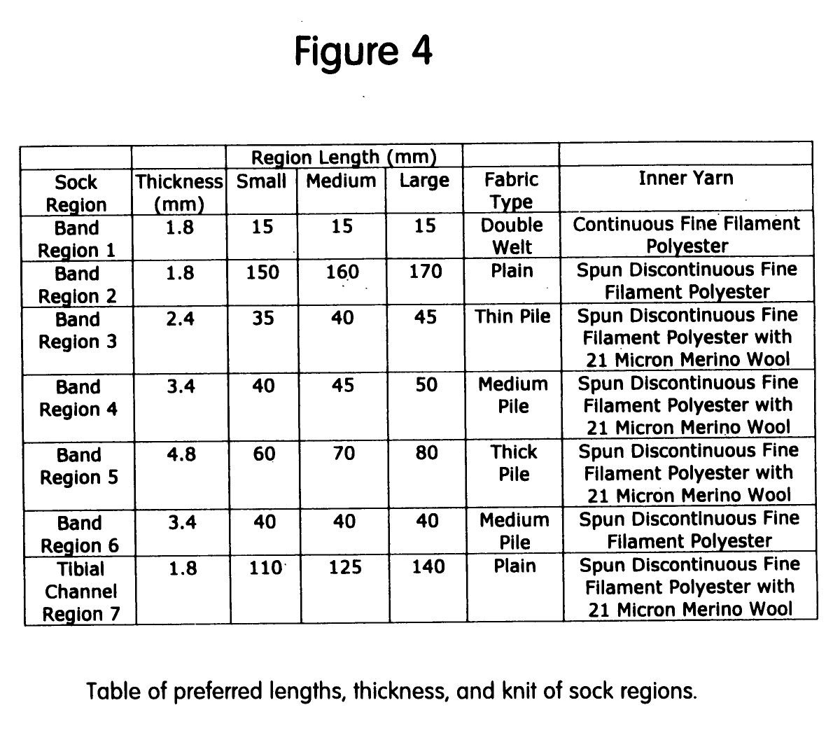

FIGS. 3-4 further show specific dimensions to be used in preferred embodiments of the present invention. FIG. 3 illustrates the dimensions provided in the above disclosure for the most preferred embodiment of the present invention, while FIG. 4 provides dimensions for three sizes of present invention sock identified as small, medium, and large, with each having different lengths in bands 1-6. Although in the examples provided in FIG. 4, the thickness dimension of the bands 1-6 does not vary with sock size, the thickness dimension can vary for differing applications.

It is understood that alterations, modifications and/or additions may be made to the present invention sock design without departing from the ambit of the invention described herein above. Thus, above-described embodiments are intended to be examples of the present invention and numerous modifications, variations, and adaptations may be made to the particular embodiments of the invention without departing from the scope and spirit of the invention, which is defined in the accompanying claims.

What is claimed is:

1. A prosthetic sock for an amputee's residual limb comprising an integral thickness which is greater over the distal end of the residual limb and tapers in graded thickness reduction proximally over the residual limb to provide increased loading to the amputee limb remnant soft tissues while reducing the loading over the amputee's tibial crest area which is more sensitive to pressure.

2. The prosthetic sock of claim 1 wherein said greater integral thickness over the distal end of the residual limb further comprises a knitted loop construction.

3. The prosthetic sock of claim 2 wherein said knitted loop construction is formed from materials selected from a group consisting of cotton, wool, polyester, and elastomeric fibres.

4. The prosthetic sock of claim 1 further comprising a substantially thinner anterior section over the area of the tibial crest of the amputee's limb remnant.

5. The prosthetic sock of claim 4 wherein said substantially thinner anterior portion has a width dimension limited to the tibial crest area of the amputee's limb remnant.

6. The prosthetic sock of claim 4 wherein said greater integral thickness over the distal end of the residual limb further comprises a knitted loop construction and wherein knitted loop construction abuts said substantially thinner anterior portion.

7. The prosthetic sock of claim 1 having five regions of graded thickness positioned to provide tapering thickness reduction over the distal and proximal ends of the residual limb.

8. The prosthetic sock of claim 1 further comprising a soft, compliant and flexible interface that assumes the shape of an amputee's residual limb and is configured so that sensitive skin tissues in the residual limb are not exposed to local pressure points and abrasion.

9. The prosthetic sock of claim 1 further comprising an inner sock surface with moisture transport capability that allows wicking of moisture away from the skin.

10. The prosthetic sock of claim 1 further comprising an outer sock surface with a smooth and durable surface that absorbs and holds moisture away from the residual limb remnant.

11. A prosthetic sock for an amputee having a residual limb with a tibial crest, said prosthetic sock comprising:
a knitted sock member of soft material having an anterior portion, a proximal portion with an open end, and a distal portion having a closed end;
said distal portion having a greater thickness dimension than said proximal portion, with an area of graduated thickness existing between said open end and said closed end that comprises a plurality of staged thickness reduction regions; and
said anterior portion of said knitted sock also having an area of thin knitting adjacent to the sensitive tibial crest area of the amputee's limb that is configured to reduce the pressure locally over the tibial crest area.

12. The prosthetic sock of claim 11 wherein said anterior portion has a width dimension limited to the tibial crest area of the amputee's limb remnant.

13. The prosthetic sock of claim 11 wherein said distal portion further comprises a knitted loop construction.

14. The prosthetic sock of claim 13 wherein knitted loop construction abuts said anterior portion.

15. The prosthetic sock of claim 11 having five of said staged thickness reduction regions.

16. The prosthetic sock of claim 11 further comprising a soft, compliant and flexible interface that assumes the shape of an amputee's residual limb and is configured so that sensitive skin tissues in the residual limb are not exposed to local pressure points and abrasion.

17. The prosthetic sock of claim 11 further comprising an inner sock surface with moisture transport capability that allows wicking of moisture away from the skin.

18. The prosthetic sock of claim 11 further comprising an outer sock surface with a smooth and durable surface that absorbs and holds moisture away from the residual limb remnant.

19. A method of manufacturing a prosthetic sock for an amputee's residual limb comprising the steps of:
providing bands of soft knitted material having different thickness dimensions; and
assembling said bands into a sock member having an integral thickness which is greater in the portion of said sock member to be positioned over the distal end of the residual limb and which also has a tapering graded reduction of thickness moving proximally toward the portion of said sock member to be positioned over the proximal end of the residual limb so as to provide increased loading to the amputee limb remnant soft tissues while reducing the loading over the amputee's tibial crest area which is more sensitive to pressure.

20. The method of claim 19 wherein said step of providing bands of soft knitted material having different thickness dimensions further comprises the providing of an anterior band having a substantially thinner soft knitted construction, and further said step of assembling said bands into a sock member further comprises the assembling of said anterior band so that when said sock member is worn over an amputee's residual limb said anterior band is adjacent to the sensitive tibial crest area of the amputee's limb and reduces the pressure locally over the tibial crest area.
